(19) United States
(12) Patent Application Publication
(10) Pub. No.: US 2003/0013185 A1
(43) Pub. Date: Jan. 16, 2003

(54) Electro-optical detection device

(76) Inventor: Ravi Saraf, Blacksburg, VA (US)

Correspondence Address: WHITHAM, CURTIS & CHRISTOFFERSON, P.C., 11491 SUNSET HILLS ROAD, SUITE 340, RESTON, VA, 20190, US

(21) Appl. No.: 10/170,209

(22) Filed: Jun. 13, 2002

Related U.S. Application Data
(60) Provisional application No. 60/299,416, filed on Jun. 21, 2001.
(60) Provisional application No. 60/368,956, filed on Apr. 2, 2002.

(51) Int. Cl. C12M 1/34; C12Q 1/00

(52) U.S. Cl. 435/287.2; 205/777.5

(57) ABSTRACT

A device for detecting molecular binding events rapidly and at very high sensitivity is provided. The device molecular binding material (such as DNA or protein) positioned in a conductive path between electrodes. Binding of a ligand to the binding material is detected as a change in a frequency response in applied oscillatory field. The device may also contain an optical measurement system (based, for example, on a laser) which detects the change in thickness of the molecular binding material under an applied oscillatory field with and without a bound material of interest.

This application claims benefit of U.S. provisional patent application serial No. 60/299,416 filed Jun. 21, 2002, and U.S. provisional patent application serial No. 60/368,956, filed Apr. 2, 2002, the complete contents of which are hereby incorporated by reference.

This invention was made using funds from grants from the Naval research foundation having grant number N00014-01-1-0977. The government may have certain rights in this invention.

### DESCRIPTION

### BACKGROUND OF THE INVENTION

1. Field of the Invention

The invention generally relates to an electro-optical device for the detection of molecular binding events. In particular, the invention provides an electro-optical device that measures the change in an electromechanical property of an immobilized molecule as it is exposed to a bioagent or other chemical, and methods for its use.

2. Background of the Invention

The detection of molecular binding events forms the basis of many fundamental investigations with wide-ranging applications, such as genetic studies and drug discovery. For example, the decipherment of DNA sequences is very important for the diagnosis of diseases, for drug design and in fostering the understanding of various biological mechanisms. Traditional methods involve “reading” a gene sequence base-pair by base-pair. DNA “chip” technologies have provided methods whereby several base-pairs can be read simultaneously. Further, this technology is a combinatorial approach in which 10's or even 100's of gene sequences can be read at the same time. However, these methods rely on tagging the DNA with a fluorescent dye to facilitate detection. This requires the utilization of labeling protocols, and results in chemical modification of the DNA, i.e. the DNA bears a fluorescent dye molecule.

Similarly, the analysis of proteins, for example in drug design, may require the investigation of enzymatic activity by probing the binding of substrates and inhibitors of an enzyme. In probing certain large proteins (such as immunoglobulins) some spectroscopic methods are available where no tagging is required (e.g. Surface Plasmon Resonance Spectroscopy). However, these techniques are not useful for the analysis of smaller proteins or for understanding the roles of specific amino acid sequences due to a lack of sensitivity, and other means, such as labeling, must be resorted to in order to detect binding products. Again, this necessitates the use of labeling protocols and results in the chemical modification of whichever component of the system is labeled.

It would be highly beneficial to have available methodology that would allow the quantitative analysis of small biomolecular binding events such as ssDNA hydridization and protein-protein and protein-nucleic acid interactions, without the necessity of chemically modifying the reactants by labeling.

### SUMMARY OF THE INVENTION

The present invention provides a device for detecting molecular binding events rapidly and at very high sensitivity. The device comprises a capacitor with at least two spaced apart electrodes; a molecular binding material positioned in a conductive path between the electrodes; a means for applying an oscillatory field to the molecular binding material using the capacitor; and a means for detecting binding of a material of interest to the molecular binding material. The detection is based on changes in a frequency response of the molecular binding material under the applied oscillatory field.

The device may also contain an optical measurement system which detects the change in thickness of the molecular binding material under an applied oscillatory field with and without a bound material of interest. Differences between the change in thickness with and without a bound material indicate that binding of the material of interest has occurred.

The optical measurement system may include a laser focused on the molecular binding material, a detector for detecting reflections from first and second opposing sides of said molecular binding material, and instrumentation for determining the thickness of the molecular binding material based on reflections detected by the detector.

### BRIEF DESCRIPTION OF THE DRAWINGS

FIG. 1. A schematic representation of the electro-optical device of the present invention.

FIG. 2A and B. A, monolayer of biomolecules on an inert, solid substrate. The monolayer may be a covalently bonded, self-assembled or simply adsorbed thin film. The film thickness at electric field E=0 is d. B, as the electric field is applied parallel to the substrate surface, the molecule in the film orient causing a change in film thickness by Δd, due to the Poisson effect.

FIG. 3. Set-up of the differential interferometer.

FIG. 4. Schematic of the signal from the spectrum analyzer. The x-axis is frequency and the y-axis is amplitude.

FIG. 5. Schematic of a biochip to perform combinatorial analysis.

### DETAILED DESCRIPTION OF THE PREFERRED EMBODIMENTS OF THE INVENTION

The present invention provides an electro-optical device for detecting and measuring changes in a macromolecule, and methods for its use. In a preferred embodiment, the change that is detected is the result of ligand binding. The device does not require the use of chemical labels; thus, the molecules that are detected can remain in an unmodified state, i.e. the method is non-destructive.

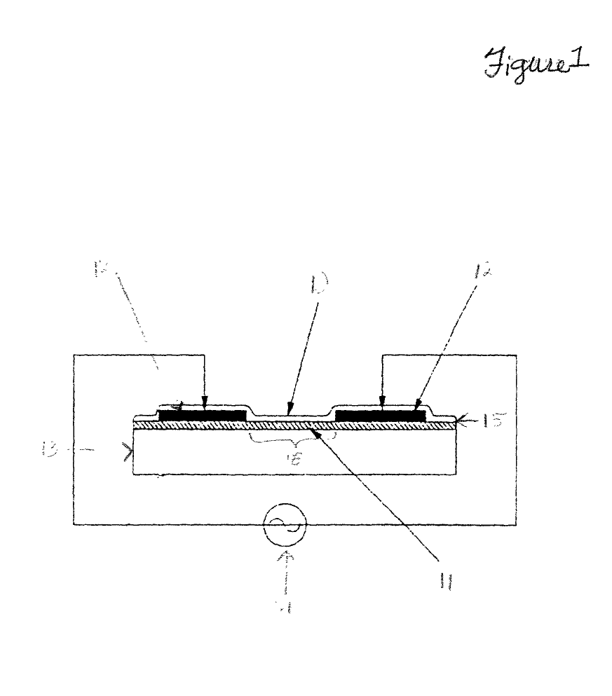

The construction of the device takes advantage of the piezoelectric properties of macromolecules when they are placed in an electric field. A schematic representation of a preferred embodiment of the device of the present invention is given in FIG. 1. With reference to FIG. 1, the device comprises a substrate 13 having a thermal layer 11 disposed thereon. Electrodes 12 are located on a top surface 15 of said thermal layer 11. The electrodes are separated by a space 16 that is wide enough to allow placement of a sample layer or film 10 therebetween. The sample film 10 may be deposited between the electrodes 12, fully covering the space 16 between the electrodes 12. The layer or film may be located between the electrodes and extend past the space 16 to conformally coat the electrodes as well. The device further comprises a power source 14 which modulates an electric field at frequency ωₛ. Only the film between the electrodes experiences the electric field. While FIG. 1 shows a pair of capacitor plates as electrodes 12, it should be understood that a comb configured capacitor arrangement may be used in the practice of this invention.

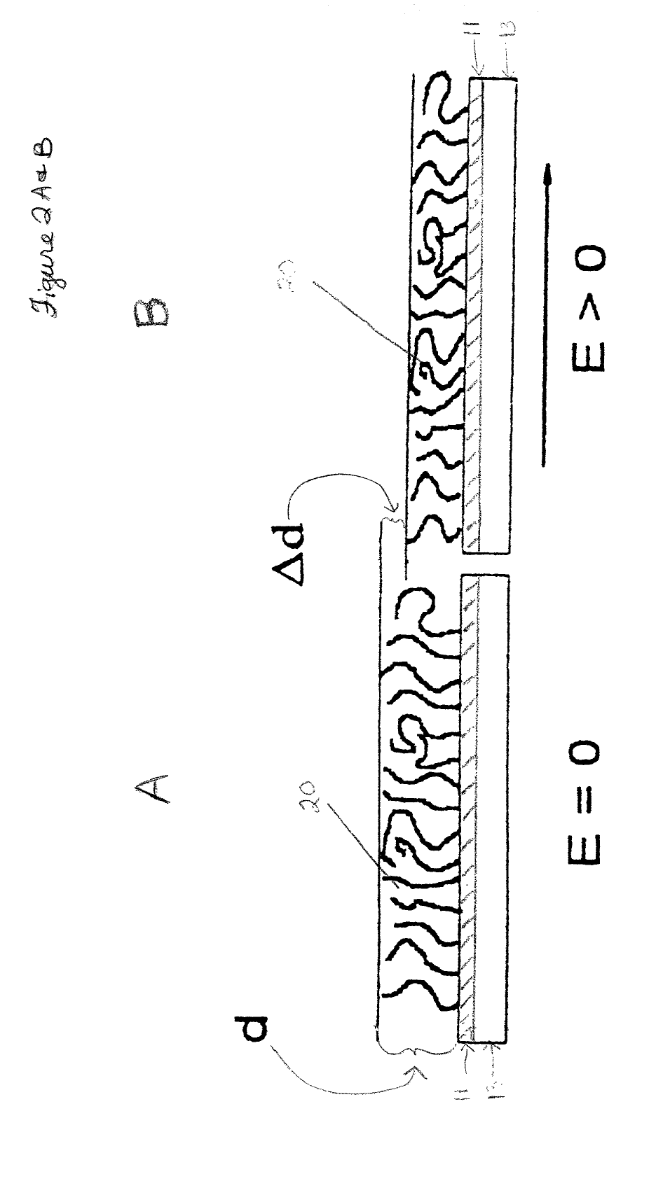

FIG. 2 illustrates the central principle of the device of the present invention. The polymeric macromolecules deposited on the device of the present invention are “mechanically soft” (i.e. non-rigid, deformable) dielectric polymers with high permanent dipole moments or polarizability. When an electric field is applied to a thin film of such polymers, they orient along the direction of the field, causing a change in the thickness of the film. FIG. 2A depicts a polymer film 20 film deposited on the thermal layer 11 disposed on a substrate 13. In the absence of an applied electric field (E=0, FIG. 2A), the polymer film exhibits a thickness d. When an electric field is applied to the polymer film 20 (E>0, FIG. 2B) the polymers orient themselves along the field direction, thereby changing the thickness of the film by Δd. If the field is oscillatory, at frequency ωₛ, the thickness will modulate at ωₛ (linier effect due to Piezoelectric behavior) and at 2ωₛ (non-linear effect due to electrostrictive behavior. For the linier effect the change in thickness, Δd, is proportional to the electric field E. For the non-linier effect the change in Δd is proportional to E². Thus, if the applied electric field is Ecos(ωt) then Δd will be proportional to Ecos(ωt), or in other words the thickness will modulate at frequency ω (piezoelectric effect). On the other hand, for the electrostrictive effect, Δd will be proportional to cos²(ωt) causing the thickness to modulate at cos(2ωt) due to the relationship that states, cos²(ωt)=1/2(1+cos(2ωt). The frequency response of the film i.e. Δd as a function of ωₛ, will depend on the coupling between the mechanical property and electronic property of the film. The electromechanical coupling will depend on the conformation, environment, and structure of the individual molecules of the film. Further, when the polymers which make up the film undergo a change (e.g. when a ligand is bound) the frequency response of the film (Δd as a function of ωₛ) will change. The device and methods of the present invention are designed to detect and measure this change.

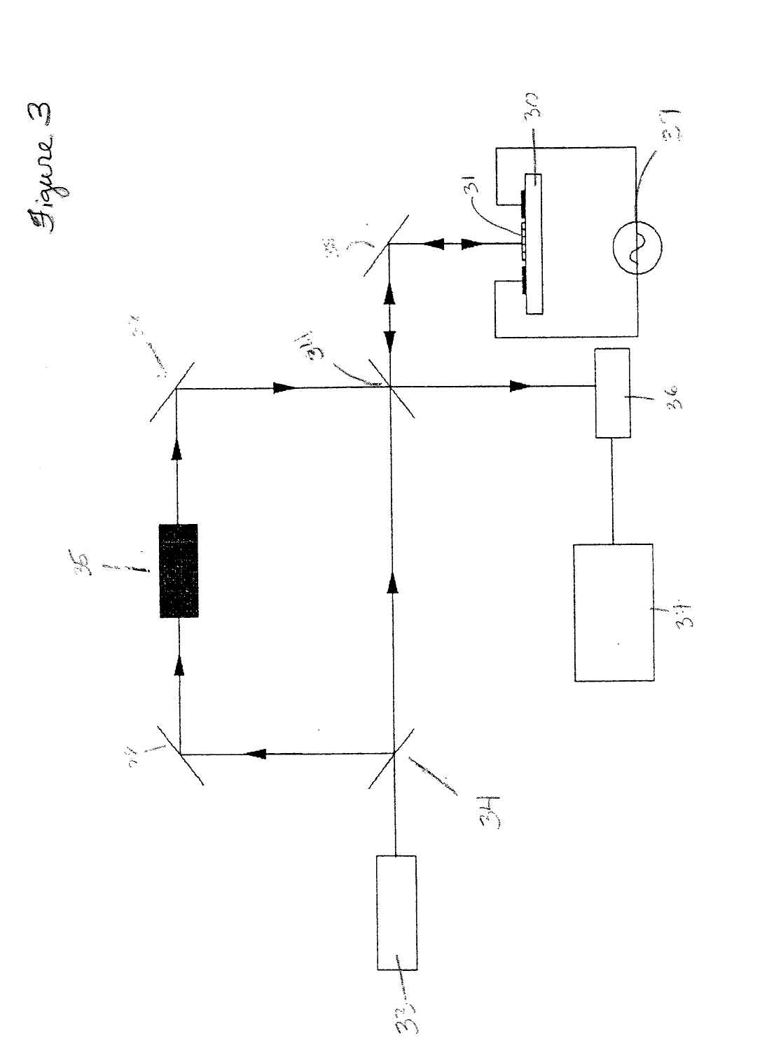

FIG. 3 illustrates a setup of a differential interferometer incorporating the device of the present invention. With reference to FIG. 3, a device of the present invention 30 with a sample film 31 deposited thereon is shown. The sample is placed under a laser beam from a laser 33. A portion of the beam is diverted via a beam splitter 34 to an acusto-optical device 35 to create a ω_{b} phase modulated reference beam. The other portion travels into the sample film 31 and is reflected back into the sample film again by the substrate 30. As the sample thickness changes due to modulation caused by the electric field, the travel distance of the laser beam changes by an amount Δ(nd), where n is the refractive index of the beam. This modulation causes a phase modulation of this portion of the laser beam by (4π/λ)(Δ(nd)), where λ is the wavelength of the laser light. Since (Δ(nd) modulates at frequencies ω and 2ω, the phase modulates at the same frequency. Upon mixing the sample beam with the reference beam into a detector 36, a frequency-modulated signal is produced. The frequency distribution is analyzed in a spectrum analyzer 37 that will show a strong peak at ω_{b} and satellite peaks at ω_{b}+/−nω Usually only n=1 and n=2 are observed.

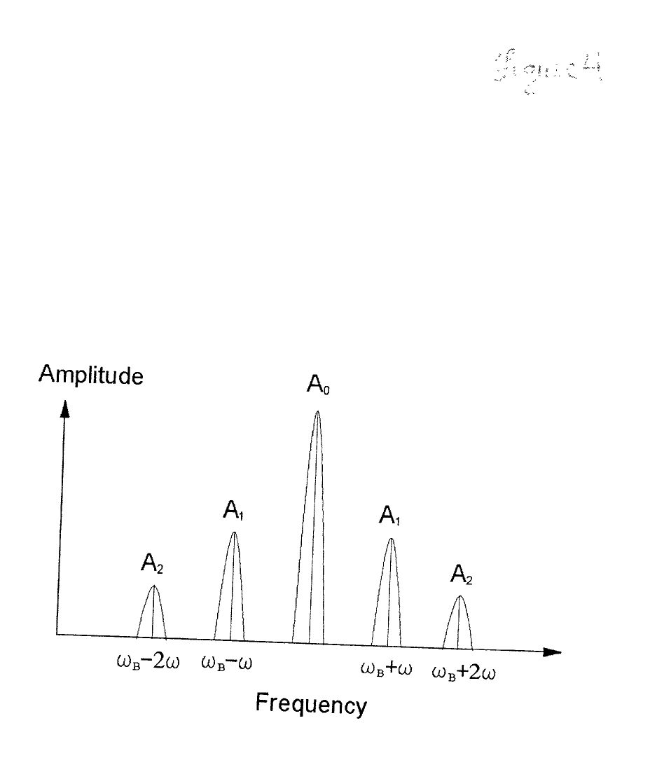

A schematic spectrum as seen on the spectrum analyzer is depicted in FIG. 4. The main peak in the center is at ω_{b} and has am amplitude of A₀. The satellite peaks at ω_{b}+/−ω are of nominally the same amplitude A₁, and the satellite peaks at ω_{b}+2ω are of nominally the same amplitude A₂. The ratio of powers with respect to the main peak to a very good approximation (within less than 0.5%) is given as:

R₁=(A₁/A₀)(2π/λ)(Δ(nd)_{I}

and

R₂=(A₂/A₀)(π/λ)(2Δ(nd)_{II}

where (Δ(nd)_{I} and Δ(nd)_{II} are thickness modulation at frequencies ω and 2ω. From the above equations, since R₁ and R₂ are measured, the thickness modulation can be determined. The material property (i.e., Δ(nd)_{I} and Δ(nd)_{II}) in the device can be measure as a function of experimental conditions such as: ω_{b}, ω, electric field amplitude, and temperature. Both the magnitude and dependence of the material properties on the experimental conditions will be sensitive to the molecules constituting the film. Thus, by measuring Δ(nd)_{I} and Δ(nd)_{II} before and after a bioreaction (e.g. a conformational change, or ligand binding) the occurrence of that particular event can be probed.

Due to the change in mass and conformation, the frequency spectrum of the film will change.

In a preferred embodiment of the present invention, the substrate portion of the device is made from a highly reflective material. In a preferred embodiment, the substrate is comprised of silicon (Si). However, those of skill in the art will recognize that other materials may also be utilized to form the substrate, including but not limited to polymers and biomolecules. In a preferred embodiment, the thermal layer is comprised of SiO₂. However, those of skill in the art will recognize that other materials may also be utilized to form the substrate layer, including but not limited to Au or Ag coated solid substrate such as glass, ceramic or polymer.

Methods of constructing such substrates and thermal layers are well known to those in the art.

In the device of the present invention, electrodes are located on a top surface of the substrate. In a preferred embodiment of the present invention, the electrodes are gold electrodes. However, those of skill in the art will recognize that other appropriate materials exist which may also be utilized to form the electrodes, including but not limited to Ag, Pt, metal alloy of noble metal, Indium-Tin-Oxide, etc.

Those of skill in the art are well-acquainted with techniques for forming electrodes on a substrate. Such methods include but are not limited to sputter deposition, vapor deposition, electro plating, electroless plating, inkjet printing, etc.

In the device of the present invention, a meso-scale layer or film of immobilized polymeric molecules is located on a top surface of the device. The polymers which are deposited on the device of the present invention are, in general, macromolecular in nature, i.e. they are in the size range of from about 100 microns to about 1 nm. The polymers have intrinsically high permanent dipole moments, or are polarizable, and they exhibit piezoelectric and electrorestrictive properties. The molecules may be capable of binding a ligand. Polymers which may be deposited as a layer or film in the practice of the present invention include but are not limited to nucleic acids (e.g. ssDNA, dsDNA, ssRNA, dsRNA, PNA), proteins (e.g. enzymes, antibodies), lipids, polysaccharides, etc. In a preferred embodiment, the polymer is DNA. In yet another preferred embodiment, the polymer is a protein.

By “deposited as a layer or film” we mean that a layer of molecules is attached to an upper surface of the device, e.g. to the thermal layer, and optionally, to the electrodes. The attachment may be effected by any of many suitable means which are well-known to those of skill in the art, including but not limited to covalent, ionic, hydrophobic bonding, adsorption, self assembly (Reference: Y Xia, J. A. Rohers, K. E. Paul, G. M. Whitesides, Unconventional Methods for Fabricating and Patterning Nanostructures, Chem. rev. Vol. 99, pages 1823-1848 (1999). The means of carrying out such attachments are well-known to those of skill in the art. For example, an SiO₂ thermal layer can be functionalized to contain hydroxyl groups using a standard Piranha solution, followed by silane treatment to obtain amine, carboxylic or other reactive groups. These reactive groups can then be utilized to bind functional groups on the molecules to be attached, e.g. with hydroxyl or phosphate groups of nucleic acids, with amine or carboxylic acid groups of proteins, by hydroxyl groups of saccharides, and the like. The density of the polymers in the film will be on the order of about 1 gm/ml. Further, the molecule may be attached directly to the top surface of the device, or may be attached via a polymeric linker or spacer. In a preferred embodiment, a single type of molecule is deposited on the device. However, for certain applications it may be desired to deposit two or more different molecules, for example, in a microarray where different types of molecules will be immobilized at different areas of the same substrate.

By “capable of binding a ligand” we mean that the polymers that comprise the film are capable of binding to another molecule of interest, i.e. they may bind to a ligand. Therefore, one potential change that may be detected by the device and methods of the present invention is the binding of a ligand by the polymers that comprise the film. Ligands which may bind to the polymers include but are not limited to complementary ssDNA, dsDNA, complementary ssRNA, dsRNA, proteins, polypeptides, lipids, saccharides, various protein substrates and inhibitors, co-factors, metals, toxic substances, small organic molecules (e.g. molecular weight less than about 100), drugs, disease producing entities (e.g. viruses, bacteria and other pathogens, or components thereof), antibodies, etc.

The source of such ligands may be any of a wide variety of sources, including but not limited to biological samples such as blood, urine, etc.; environmental samples such as water from reservoirs or waste water; comestible items; and the like. Further, the device of the present invention may function in either a liquid environment, or in air.

In another embodiment of the invention, changes other than the binding of a ligand are detected by the device and methods of the present invention. For example, changes in the conformation of an immobilized macromolecule as a result of an alteration in the environment of the macromolecule may also be detected. Such alterations include but are not limited to changes in pH, temperature (e.g. to detect denaturation and/or renaturation, sensitivity to cold, etc), ionic strength, etc.

Further, the detection of the impact of an alteration in the environment on the binding of a ligand may be detected. For example, the ability of a macromolecular polymer to bind a ligand at different pH values, at different ionic strengths, at different temperatures, or in the presence of other effector molecules, may also be detected by the device and methods of the present invention. Those of skill in the art will recognize that any alteration in the polymeric macromolecules deposited on the device of the present invention may be detected by the device and methods of the present invention, so long as the alteration results in a measurable change in the piezoelectric properties of the polymers in the film.

In one embodiment of the invention, the change may be induced by ligand binding. By “ligand binding” we mean that a ligand has become associated with polymers in the polymer film. The association may be irreversible or reversible, and may be the result of binding via, for example, covalent, ionic, or hydrophobic forces. If the association is reversible, the affinity of the ligand for the polymer will generally be on the order of about 1% to about 100%.

Binding events which may be detected by the device and methods of the present invention include but are not limited to nucleic acid hybridization (e.g. complementary ssDNA and/or ssRNA binding); protein-ligand binding (e.g. protein-substrate or protein-inhibitor binding); the binding of regulatory factors to a macromolecule such as a protein; and the like.

With respect to ligand binding, the values of the amount of change that is detected will depend on the “perfection” of binding. For example, for ssDNA hybridization, the location of one or more base-pair mismatches can be detected. Thus, by measuring the above ratios, subtle variations in sequence (for example, those caused by mutations) can be determined. Although in principle the ratios may be estimated theoretically for various sequences with corresponding mismatches, the values may more simply be obtained by experimental calibration. In this case, ratios of R1 and R2 may be determined experimentally, and a database for known sequences with a predetermined number and location of mismatches may be established from the data.

The device and methods of the present invention detect molecular binding events rapidly (the data acquisition response time is<1 minute) and at extremely low sensitivity.

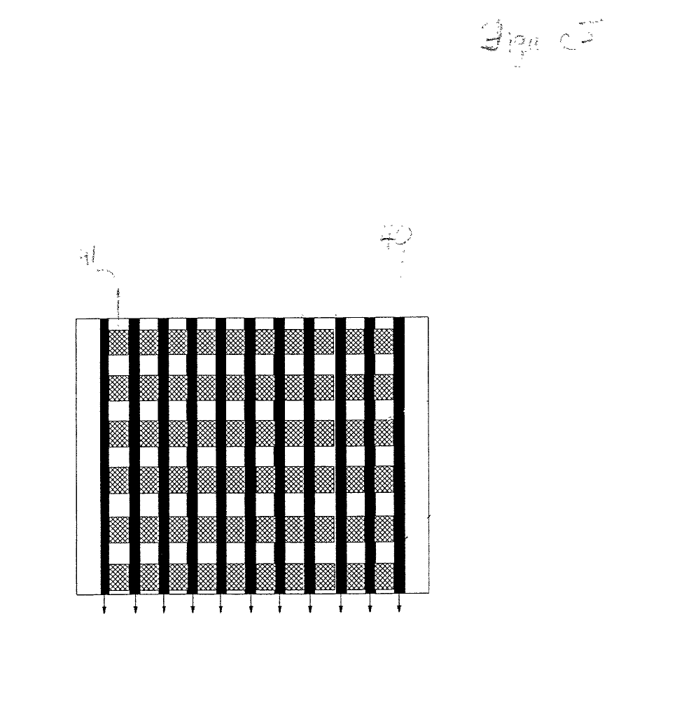

In one embodiment of the present invention, the device of the present invention is integrated into a chip format that allows combinatorial analysis. Such an arrangement is depicted in FIG. 5 where parallel electrode lines 40 of width w can be deposited at spacing s and patches of sample film 41 (pixels) can be deposited between the electrode lines 40. An electric field is applied and a laser beam is scanned over various locations on the chip to probe the frequency response to the electric field. The patches of sample film 41 may be the same (for example, to provide control sections on the chip) or different (for example, so that many different molecules, such as ssDNA sequences, may be assayed on a single chip). The minimum value of s will be determined by the size of the laser beam, and in general, pixel size will be in the range of about 10 by 10 μm or less. Thus, a 1 centimeter square chip with interconnection pads,(i.e. pads that connect the electrode to the power supply, which can themselves be electrodes) can readily contain more than 3,000 pixels, i.e. can contain more than 3000 different patches of immobilized molecules. For a typical DNA chip, the pixel size is determined by the size of the probe beam. Typically, for a probe beam from an He—Ne laser with λ=633 nm, the pixel may be about 5 by 5 μm.

Those of skill in the art will recognize that the device and methods of the present invention may be utilized for the detection of numerous substances in a wide variety of fields. For example, the device and methods are useful in fields of drug discovery to identify compounds that bind to macromolecules (e.g. inhibitors of an enzyme); or to accomplish DNA sequencing via hybridization of ssDNA of varying sequences; or to detect pollutants, toxins and other noxious substances, for example, in biological warfare.

### EXAMPLES

### Example 1

### Detection of ssDNA Hybridization

Two electrodes are established a short distance (e.g. 10 microns) apart. Between the electrodes, a film of ssDNA of relatively short length (e.g. 25 bp) is immobilized and kept in an extended form via “optical tweezers”. An electric field is applied between the electrodes, causing the DNA to become a charged material in which one side will be positive and will move toward the negative electrode. Upon reversing the polarity of the field, the DNA will appear to “dance” in a similar fashion to long grass waving in the wind, resulting in a measurable change in the thickness of the film. The electric field is oscillated (vibrated) at different frequencies. At certain frequencies, the motion of the DNA is greater because it is at resonance at those frequencies.

The ssDNA resembles a piece of rope, with its vibrational behavior dependent on the rigidity of the rope. If a binding event occurs, such as the hybridization of a complementary strand of ssDNA, the rope will be substantially thicker and more rigid than the ssDNA. This causes a change in the resonance frequency of the molecules in the film. When such a change is detected, it is indicative of a binding event.

Measurements as precise as to the order of a single base pair are made and the resulting frequency changes are used to quantify the degree of hybridization of the known ssDNA sequence. For example, if a known ssDNA of 25 bp joins to complementary ssDNA in a biological sample, if of the 25 bp 24 are exactly matched but 1 is mismatched, the structure is slightly different than if all 25 bp are exactly matched. Therefore, the oscillation of the 24 out of 25 bp matched double helix (or any other combination, e.g. a 23 out of 25 bp match) differs from that of the perfect 25 bp match (or from any other bp match combination.) Thus, the degree of hybridization is detected by detecting the corresponding changes in frequency. Moreover, because changes in vibration on the order of 0.5 angstroms are detected, and a bp is about 3 angstroms in size, the device of the present invention also locates unbound sites such as at the end or middle of the known ssDNA sequence.

While the invention has been described in terms of its preferred embodiments, those skilled in the art will recognize that the invention can be practiced with modification within the spirit and scope of the appended claims. Accordingly, the present invention should not be limited to the embodiments as described above, but should further include all modifications and equivalents thereof within the spirit and scope of the description provided herein.

We claim:

1. A device for detecting molecular binding events, comprising:
a capacitor with at least two spaced apart electrodes;
a molecular binding material positioned in a conductive path between said at least two spaced apart electrodes;
means for applying an oscillatory field on said molecular binding material using said capacitor; and
means for detecting binding of a material of interest to said molecular binding material based on changes in a frequency response of said molecular binding material under an applied oscillatory field.

2. The device of claim 1 wherein said means for detecting binding comprises an optical measurement system which detects a first change in thickness of said molecular binding material under an applied oscillatory field without a bound material of interest and a second change in thickness of said molecular binding material under an applied oscillatory field with a bound material of interest, wherein differences between said first and second change in thickness indicate binding of said material of interest.

3. The device of claim 2 wherein said optical measurement system comprises a laser focused on said molecular binding material, a detector for detecting laser light which has passed through or been reflected by said molecular binding material and a control laser light which has not passed through or been reflected by said molecular binding material, and instrumentation for determining a thickness of said molecular binding material based on differences in measurements between said laser light and said control laser light.

4. A device for detecting molecular binding events, comprising:
a substrate;
a plurality of capacitors formed on said substrate, each of said capacitors having at least two spaced apart electrodes;
a molecular binding material positioned in a conductive path between each of said at least two spaced apart electrodes;
means for applying an oscillatory field on said molecular binding material using said capacitor; and
means for detecting binding of a material of interest to said molecular binding material based on changes in a frequency response of said molecular binding material under an applied oscillatory field.

5. The device of claim 4 wherein said means for detecting binding comprises an optical measurement system which detects a first change in thickness of said molecular binding material under an applied oscillatory field without a bound material of interest and a second change in thickness of said molecular binding material under an applied oscillatory field with a bound material of interest, wherein differences between said first and second change in thickness indicate binding of said material of interest.

6. The device of claim 5 wherein said optical measurement system comprises a laser focused on said molecular binding material, a detector for detecting laser light which has passed through or been reflected by said molecular binding material and a control laser light which has not passed through or been reflected by said molecular binding material, and instrumentation for determining a thickness of said molecular binding material based on differences in measurements between said laser light and said control laser light.

7. The device of claim 4 wherein said molecular binding material includes a plurality of different materials, each of said different materials being positioned between spaced apart electrodes of different capacitors of said plurality of capacitors.

8. A method for detecting molecular binding events, comprising the steps of:
depositing a sample between spaced apart electrodes of a capacitor, wherein said capacitor includes a molecular binding material positioned in a conductive path between said at least two spaced apart electrodes;
applying an oscillatory field on said molecular binding material using said capacitor; and
detecting binding of a material of interest in said sample to said molecular binding material based on changes in a frequency response of said molecular binding material under an applied oscillatory field.

9. The method of claim 8 wherein said detecting step includes detecting a first change in thickness of said molecular binding material under an applied oscillatory field without a bound material of interest and a second change in thickness of said molecular binding material under an applied oscillatory field with a bound material of interest, wherein differences between said first and second change in thickness indicate binding of said material of interest.
